(19) United States
(12) Patent Application Publication
(10) Pub. No.: US 2010/0297036 A1
(43) Pub. Date: Nov. 25, 2010

(54) PRESSURIZED COSMETIC COMPOSITION COMPRISING AT LEAST ONE ANIONIC POLYURETHANE ELASTOMER AND AT LEAST ONE PROPELLANT

(76) Inventor: Laëtitia FEUILLETTE, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/470,819

(22) Filed: May 22, 2009

(51) Int. Cl. A61K 8/87; A61Q 5/06

(52) U.S. Cl. 424/47

(57) ABSTRACT

Provided is a cosmetic composition packaged in an aerosol device comprising in a cosmetically acceptable medium, at least one film-forming anionic polyurethane elastomer and at least one propellant. Also provided is a method for shaping the hair comprising applying this cosmetic composition to the hair.

Provided is a pressurized cosmetic composition comprising at least one anionic polyurethane elastomer and at least one propellant.

Styling compositions, such as lacquers and sprays, packaged in the form of aerosol sprays may be composed of a lipid phase comprising, in a cosmetically acceptable alcoholic or aqueous/alcoholic medium, at least one film-forming polymer and of a propellant which is a liquefiable gas under reduced pressure partially dissolved in the liquid phase.

In the field of hair products, it is possible to attempt to manufacture lacquers as an aerosol comprising few or no volatile organic compounds, for example, for ecological reasons, while retaining good properties of shaping and of form retention of the hairstyle.

The current fashion in the field of hair styling is aimed at a more natural look and feel and, intermediately, at a more flexible and less constrained fixing with a durability equivalent to their normal products. In addition, there exists a very high demand on the part of consumers for styling products which would exhibit an even better hold over time than that obtained currently.

In point of fact, marketed products currently may exhibit a strong fixing, indeed even very strong fixing, which may be visible and perceptible to the touch and without true hold over time when faced with the usual mechanical stresses, such as the passage of the hands, the wearing of a helmet or the taking-off of pullovers.

For example, this problem may exist for pressurized formulations.

It is possible to obtain nonrigid and lasting fixing via the introduction of a polymer exhibiting elastic mechanical properties, such as a polyurethane.

However, in the case of aerosols, such polyurethane-based solutions may often deliver white drops onto the hair during application, due to a low solubility of the polyurethanes in the more or less aqueous solvents. In addition, styling products in the pressurized form comprising a polyurethane may exhibit a synthetic and not very natural feel.

Currently, there may exist no or few cosmetic compositions in the aerosol form which make it possible to provide the hair with elastic, flexible and lasting fixing.

Another aspect of the disclosure is that of obtaining good spraying conditions and a product which may exhibit at least one of properties of durable, elastic and flexible shaping and form retention.

The use, in pressurized hair compositions, of certain polyurethanes may result in formulations which can be easily sprayed, for example when they comprise a high proportion of water, without the formation of white drops. These compositions can be easily applied to the hair and the feel of the hair is cosmetic and nonsticky. The film-forming polyurethane elastomers may exhibit a satisfactory styling power and can be easily removed by simple shampooing, leaving clean hair. Brushing the treated hair can leave the hair soft and shiny.

For example, one feature of the polyurethanes of the disclosure is that they can be sprayed in a formulation comprising a propellant gas. They can form, before drying, transparent drops on the individual hair which may disappear on drying.

On applying such a composition to the individual hair, an elastic sheathing may be formed which may make possible flexible fixing, more natural than that introduced by conventional fixing polymers used in aerosols, and which has excellent adhesion to the hair.

The formulation can make it possible to obtain a hairstyle which may be resistant to mechanical stresses: passage of the hands through the hair, wearing of a helmet or taking-off of a pullover.

### SUMMARY

Provided is a cosmetic composition packaged in an aerosol device comprising, in a cosmetically acceptable medium, at least one film-forming anionic polyurethane elastomer (A) and at least one propellant,

the at least one film-forming anionic polyurethane elastomer (A) comprising:

(i) at least one side chain comprising at least one ethylene oxide unit, wherein the at least one side chain

represents from 12 to 80% by weight of polyurethane (A)

has a Mw ranging from 1000 g/mol to 30,000 g/mol

(ii) one main chain comprising at lest one unit derived from:

a polypropylene glycol (PPG) and optionally a second non-ionic polyol;

a dihydroxy-carboxylic acid and

at least one di-isocyanates, and optionally

a chain extender organic polyamine having an average of at least two primary amine groups.

This composition can make it possible, for example, to obtain a flexible and nonbrittle film on human keratinous substances which matches their movements.

In addition, the composition described herein may provide conditioning of the hair.

Also provided is a method for the shaping or form retention of the hairstyle comprising applying this composition to the hair.

### DETAILED DESCRIPTION

### Chemistry of the Film-Forming Polyurethane Elastomer

The film-forming anionic polyurethane elastomer (A) may be prepared according to the process described in PCT Patent Application Publications WO2006/124250 and WO03/087183.

In one embodiment, the film-forming anionic polyurethane elastomer (A) comprises

(i) at least one side chain comprising at least one ethylene oxide unit, wherein the at least one side chain

represents from 12 to 80% by weight of polyurethane (A)

has a Mw ranging from 1000 g/mol to 30,000 g/mol

(ii) one main chain comprising at least one unit derived from:

a polypropylene glycol (PPG) and optionally a second non-ionic polyol;

a dihydroxy-carboxylic acid, such as dimethylolpropionic acid (DMPA);

at least one di-isocyanate, such as isophorone diisocyanate; and optionally

a chain extender organic polyamine having an average of at least two primary amine groups.

The polyurethane elastomer (A) is soluble or dispersible, either in an aqueous medium (100% of water) or in a mixture of water/ethanol solvents comprising at most 30% by weight of ethanol. For example, in some embodiments, at least 10 grams of the polymer (A) are soluble or dispersible in 90 grams of water or of water/ethanol mixture.

The film-forming anionic polyurethane elastomer (A) may be partially neutralized, completely neutralized or overneutralized with inorganic or organic bases, for example, with sodium hydroxide and/or triethylamine, in an amount which makes it possible to, for example, obtain a degree of neutralization of the anionic functional groups of the polymer of 0 to 150%, such as, of 50 to 100%. The “150% degree of neutralization” is obtained with an amount of inorganic or organic base equal to 1.5 times the amount of base necessary to obtain a 100% degree of neutralization.

The result of this is that the film-forming polyurethane elastomer (A) is provided in the form of a salt composed of two parts: a polyurethane in the anionic form and with a metal or organic cationic counterion.

### Mechanical Profile of the Film-Forming Polyurethane Elastomer

In some embodiments, in the compositions according to the disclosure, the anionic film-forming polyurethane elastomer (A) is chosen so that the film obtained by drying this polymer (A), at ambient temperature (24° C.±2° C.) and at a relative humidity of 48%±5%, exhibits a mechanical profile defined by at least:

(a) a degree of elongation at break (εᵣ) of greater than or equal to 730%±5% (e.g., −5% of 730% represents 694%), and/or

(b) an instantaneous recovery (Rᵢ) of greater than or equal to 70%±5%, after an elongation of 150%, and/or

(c) a recovery (R₃₀₀ₛ) at 300 seconds of greater than 80% ±5%.

### Measurement of the Parameters

Within the meaning of the present disclosure, the term “film obtained by drying at ambient temperature (24° C.±2° C.) and at a relative humidity of 48%±5%” is understood to mean the film obtained, under those conditions, from a mixture comprising 6% of film-forming elastomeric polymer (A) active material (a.m.) in a solvent (S). The amount of active material is meant relative to the total weight of solvent (S) and the amount of mixture is adjusted in order to obtain, in a Teflon matrix, a film with a thickness of 500 μm±50 μm after drying of the mixture, this drying being continued until the weight of the film no longer changes, which generally represents 10 days.

The solvent (S) is the ethanol/water mixture in a 30/70 ratio by weight.

Within the meaning of the present disclosure, the “degree of elongation at break” and the “degree of recovery” are evaluated by means of the tests described below.

In order to measure the instantaneous recovery and the recovery at 300 seconds, the polymer film obtained is cut into test specimens of rectangular shape with a length of 80 mm and a width of 15 mm.

In order to measure the elongation, the polymer film obtained is cut into test specimens having the shape of a dumbbell.

The tests may be carried out, on a device sold under the Lloyd name or sold under the Zwick name, under the same temperature and humidity conditions as for the drying, that is to say a temperature of 24° C.±2° C. and a relative humidity of 48%±5%.

The test specimens are drawn at the rate of 20 mm/min and the distance between the clamping jaws is 50 mm±1 mm.

The degree of elongation at break represents the ratio of maximum elongation of the film before it breaks to its initial length measured before having undergone the strain.

The procedure for determining the instantaneous recovery (Rᵢ) is as follows:

the test specimen is drawn by 150% (εₘₐₓ), that is to say 1.5 times its initial length(I₀),

the stress is released while imposing a return rate equal to the pull rate, e.g., 20 mm/min, and elongation of the test specimen is measured as a percentage, after returning to zero load (εᵢ).

The instantaneous recovery in % (Rᵢ) is given by the formula below:

Rᵢ=((εₘₐₓ−εᵢ)/εₘₐₓ)×100

In order to determine the recovery at 300 seconds, the test specimen which has been subjected to the preceding operations is maintained at zero stress for an additional 300 seconds and its degree of elongation is measured as a percentage (ε₃₀₀ₛ).

The recovery at 300 seconds in % (R₃₀₀ₛ) is given by the formula below:

R₃₀₀ₛ=((εₘₐₓ−ε₃₀₀ₛ)/εₘₐₓ)×100

In the compositions in accordance with the disclosure, the film-forming polyurethane elastomer (A) or the blend of anionic film-forming polyurethane elastomers (A) is, for example, present at a concentration ranging from 0.05% to 20% by weight, such as from 0.1% to 15% by weight and further such as from 1% to 10% by weight, relative to the total weight of the composition.

### Additives and Propellant

In some embodiment, the cosmetically acceptable medium comprises at least one solvent chosen from water, C₁-C₄ alcohols, such as ethanol, isopropanol, tert-butanol and n-butanol, propylene carbonate, polyols, such as propylene glycol, polyol ethers, and acetone, one exemplary solvent being ethanol.

For example, in some embodiments, the cosmetically acceptable medium is aqueous or aqueous/alcoholic, the alcohol being a C₁-C₄ alcohol.

When the alcohol is present, its proportion is, for example, from 1 to 99% by weight, such as from 5 to 80% by weight and further such as from 8 to 50% by weight, relative to the total weight of the hair treatment composition and of the propellant.

The propellant can be any liquefiable gas generally used in aerosol devices. The choice is made, for example, of dimethyl ether, C₃₋₅ alkanes, chlorinated and/or fluorinated hydrocarbons, such as 1,1-difluoroethane, and their mixtures, such as, for example, mixtures of dimethyl ether and of C₃₋₅ alkanes and mixtures of 1,1-difluoroethane and of dimethyl ether and/or of C₃₋₅ alkanes. Non-limiting use may also be made, as propellant, of carbon dioxide gas, nitrous oxide, nitrogen or compressed air or their mixtures.

For example, in some embodiments, the propellant gas used is dimethyl ether or C₃₋₅ alkanes and such as propane, n-butane and isobutane and their mixtures.

The liquid phase/propellant ratio by weight for the pressurized hair compositions of the present disclosure ranges, for example, from 50 to 0.05, such as from 50to 1.

The aerosol device used to package the compositions of the disclosure can be a two-compartment aerosol device composed of an external aerosol can comprising an internal bag hermetically welded to a valve. The composition is introduced into the internal bag and a compressed gas is introduced between the bag and the can at a pressure sufficient to bring about the departure of the product in the form of a spray through the orifice of a nozzle. Such a device is sold, for example, under the name EP Spray by EP-Spray System S.A. The compressed gas is, for example, used under a pressure of from 1 to 12 bar, such as of from 9 to 11 bar.

In the case of foam aerosols, the compositions introduced into the aerosol device can, for example, be provided in the form of lotions, dispersions or emulsions which, after dispensing from the aerosol device, form foams to be applied to keratinous substances.

These foams should be sufficiently stable not to rapidly liquefy and should also rapidly disappear, either spontaneously or during the massaging which causes the composition to penetrate into and/or to be distributed over the keratinous substances, such as the hair.

In the case of foam aerosols, the hair treatment composition according to the disclosure can additionally comprise at least one cationic, nonionic, anionic or amphoteric surface-active agent.

In addition, the hair treatment composition according to the disclosure can comprise at least one fatty substance (inter alia vegetable, animal, mineral and synthetic oils, resins and natural or synthetic waxes).

For example, the at least one polyurethane of the composition according to the disclosure is optionally combined with a plasticizing agent, a cosmetic agent and/or an agent which facilitates the formation of a film of the at least one polyurethane on the keratinous substances. Such agents can be chosen from any compound known to a person skilled in the art as being capable of performing the desired role. For example, these agents may be chosen from conventional plasticizers or coalescence agents, such as:

glycols and their derivatives, such as diethylene glycol ethyl ether, diethylene glycol methyl ether, diethylene glycol butyl ether, diethylene glycol hexyl ether, ethylene glycol ethyl ether, ethylene glycol butyl ether, ethylene glycol hexyl ether or pentylene glycol,

glycerol esters,

propylene glycol derivatives, such as, propylene glycol phenyl ether, propylene glycol diacetate, propylene glycol butyl ether, tripropylene glycol butyl ether, propylene glycol methyl ether, dipropylene glycol ethyl ether, tripropylene glycol methyl ether, diethylene glycol methyl ether or propylene glycol butyl ether,

esters of acids, for example, carboxylic acids, such as citrates, phthalates, adipates, carbonates, tartrates, phosphates or sebacates, and

their mixtures.

The amount of plasticizing agents and/or of film-forming agents can be chosen by a person skilled in the art on the basis of his general knowledge so as to obtain a polymer system resulting in a film having the desired mechanical properties. In practice, this amount varies from 0.01 % to 25% of the total weight of the composition, for example, from 0.01% to 15%.

The compositions according to the disclosure can also comprise other cosmetically acceptable adjuvants, such as, for example, ionic or nonionic surface-active agents, nonionic, cationic, anionic or amphoteric thickening agents, penetrating agents, fragrances, colorants, plasticizers, buffers and various conventional adjuvants, such as waxes, volatile or nonvolatile and cyclic, linear or branched silicones which are organomodified, such as alkoxylated, or modified by amine groups or unmodified, ceramides, pseudoceramides, vegetable, mineral or synthetic oils, vitamins or provitamins, such as panthenol, opacifying agents, reducing agents, emulsifiers, preservatives, inorganic fillers, pearlescent agents, glitter, sunscreens, proteins, anionic, nonionic, cationic or amphoteric fixing polymers other than the anionic polyurethane elastomers (A), moisturizing agents, emollients, softening agents, antifoaming agents, antiperspirant agents, agents for combating free radicals, bactericides, sequestering agents, antidandruff agents, antioxidants, basifying agents, acidifying agents and any other additive conventionally used in cosmetic compositions intended to be applied to the hair, for example, conditioning agents, such as cationic polymers, cations, cationic surfactants, fatty alcohols, silicone gums and resins, chitosans and their derivatives, solubilizing agents, acidifying agents, basifying agents, inorganic and organic thickening agents, such as natural or synthetic nonionic associative thickening agents, antioxidants, hydroxy acids, penetrating agents, fragrances, peptizing agents, amino acids and preservatives.

A person skilled in the art will take care to choose the optional additives and their amounts so that they do not interfere with the properties of the compositions of the present disclosure. These additives are present in the composition at an amount ranging from 0 to 20% by weight, relative to the total weight of the composition.

The application of the compositions comprising the polyurethane elastomer(s) of the disclosure in a pressurized medium can be carried out with introduction of heat, for example using a hair dryer or flat-nose tongs.

The pH of the compositions according to the disclosure is generally from 2 to 9 and such as from 3 to 8. It can be adjusted to the chosen value using basifying or acidifying agents commonly used in cosmetics for this type of application.

The following examples are intended to illustrate the present disclosure without however limiting the scope thereof.

### EXAMPLES

In the tables below, the values were expressed as percentages by weight of active material in the final formulation.

The polymers were all in the form of a dispersion in water or in an aqueous/alcohol medium.

The film-forming anionic polyurethane elastomers (A) employed in the examples were defined below by their chemical composition and their mechanical properties. The compositions of the film-forming anionic polyurethane elastomers (A) were expressed on a dry basis.

| | | Instantaneous | Recovery at |
|---|---|---|---|
| | Elongation | recovery | 300 seconds |
| A1 | 1300 | 70.5 | 90.6 |
| A2 | 833 | 74.9 | 90.8 |
| A3 | | 76.7 | 94.6 |
| A4 | 877 | 74.7 | 91.8 |
| A5 | >1200 | 84.2 | 94.2 |
| A6 | | 77.6 | 93.5 |

CHEMICAL COMPOSITIONS BY WEIGHT

| | IPDI | PEO | PPG | DMPA | TMP | HNA | PDMS | MPEG | EDA | TEA | NaOH | TOTAL |
|---|---|---|---|---|---|---|---|---|---|---|---|---|
| A1 | 28.1 | 22.7 | 26.3 | 3.0 | 0.5 | 14.5 | | | 2.7 | 2.3 | | 100.0 |
| A2 | 28.9 | 16.7 | 23.0 | 2.9 | 0.5 | 23.0 | | | 2.7 | 2.1 | | 100.0 |
| A3 | 24.6 | 15.9 | 46.4 | 2.9 | 1.6 | | 3.0 | | 3.0 | 0.9 | 1.6 | 100.0 |
| A4 | 29.1 | 14.1 | 49.7 | 2.9 | 0.5 | | | | 2.3 | 1.1 | 0.5 | 100.0 |
| A5 | 25.9 | 16.7 | 48.5 | 3.0 | | | | | 3.2 | 2.7 | | 100.0 |
| A6 | 23.8 | 15.3 | 44.4 | 2.8 | | | | 8.3 | 2.9 | 2.5 | | 100.0 |

For all polyurethane elastomers A1 to A6, the ethylene oxide side chains exhibited a molecular weight of 1200 g/mol and they represented more than 12% by weight of the polyurethane elastomer (A).

IPDI: Isophorone diisocyanate

HNA: Hexanediol neopentyl adipate polyester polyol (Mw=1000)

PPG: Polypropylene glycol (Mw=2000)

PEO: Polyethylene oxide 1,3-diol (Mw=1200)

DMPA: Dimethylolpropionic acid

TMP: Trimethylolpropane

EDA: Ethylenediamine

TEA: Trethylamine

PDMS: Polydimethylsiloxane polyol

MPEG : Methoxy polyethylene glycol

NaOH: Sodium hydroxide

EtOH: Ethanol
Lacquers and aerosol foams were prepared below.

1. Lacquer Compositions for Shaping the Hair

| Lacquer | | | | | | | | |
|---|---|---|---|---|---|---|---|---|
| compositions | | | | | | | | |
| 85% VOC | 1 | 2 | 3 | 4 | 5 | 6 | 7 | 8 |
| A1 or A2 or A3 or | 6.5 | 5.5 | 4.5 | 2 | 6.5 | 5.5 | 4.5 | 2 |
| A4 of the disclosure | | | | | | | | |
| Ethanol | 50 | 50 | 50 | 50 | 40 | 40 | 40 | 40 |
| Water | 8.5 | 9.5 | 10.5 | 13 | 8.5 | 9.5 | 10.5 | 13 |
| Dimethyl ether | 35 | 35 | 35 | 35 | 45 | 45 | 45 | 45 |
| Lacquer | | | | | | | | |
| compositions | | | | | | | | |
| 55% VOC | 9 | 10 | 11 | 12 | 13 | 14 | 15 | 16 |
| A1 or A2 or A3 or | 6.5 | 5.5 | 4.5 | 2 | 6.5 | 5.5 | 4.5 | 2 |
| A4 of the invention | | | | | | | | |
| Ethanol | 20 | 20 | 20 | 20 | 10 | 10 | 10 | 10 |
| Water | 38.5 | 39.5 | 40.5 | 43 | 38.5 | 39.5 | 40.5 | 43 |
| Dimethyl ether | 35 | 35 | 35 | 35 | 45 | 45 | 45 | 45 |

| Compositions | 17 | 18 | 19 | 20 |
|---|---|---|---|---|
| A5 of the invention | 1.7 | 1.7 | 1.7 | 1.7 |
| Ethanol | 57.8 | 20 | 47.8 | 10 |
| Eau | 5.5 | 43.3 | 5.5 | 43.3 |
| Dimethyl ether | 35 | 35 | 45 | 45 |

The aerosol device used comprised a Coster valve (1467P:gi=0.33/(i=0.33/without additional gas connection) and a Precision nozzle (DSPR128:di=0.51)

| Foam aerosol compositions | 21 | 22 | 23 | 24 | 25 | 26 |
|---|---|---|---|---|---|---|
| A1 or A2 or A3 or A4 or A5 | 4 | 1 | 0.5 | 4 | 1 | 0.5 |
| or A6 of the invention | | | | | | |
| Laureth-4 | 1.1 | 1.1 | 1.1 | 1.1 | 1.1 | 1.1 |
| Propylene glycol | 2.4 | 4 | 4 | 4 | 2.5 | 2.5 |
| Glycerol | 0.5 | 0.5 | 0.5 | 0.5 | 0.5 | 0.5 |
| Preservatives | 0.66 | 0.66 | 0.66 | 0.67 | 0.67 | 0.67 |
| Distilled water | q.s. for | q.s. for | q.s. for | q.s. for | q.s. for | q.s. for |
| | 95% | 95% | 95% | 92% | 92% | 92% |
| Isobutane/butane/propane | 5 | 5 | 5 | 8 | 8 | 8 |
| 56/24/20 | | | | | | |

The ternary propellant gas mixture used was a mixture of isobutane/butane/propane (56/24/20) sold under the name of “Aerogaz 3.2 N” by Elf Aquitane.

### Protocol for Application of the Compositions at Ambient Temperature

Undyed, unbleached and unpermed locks with a natural chestnut colour, weighing 2.7 g were used.

1. A standard shampooing was carried out,

2. the hair was rinsed with water,

3. the hair was superficially dried with a towel,

4. the composition was applied to the wet or dry lock,

5. the hair was allowed to dry in the open air or else, for example, with a hood dryer or hair dryer, or flat-nose tongs, optionally with application of a mechanical action, for example using a brush (blow drying).

### Results

In the case of the lacquers, visible drops were observed on the individual hair on application which were transparent, in contrast to normal aerosol compositions comprising a polyurethane.

In the case of the foams, the foam dispensed by the aerosol was opaque, white, creamy and sufficiently expanded.

For each of the compositions (foam or lacquer), significant shaping of the hair and a hold of this shaping were obtained. The fixing of the hair was elastic and flexible. The polymer film formed was nonfriable and thus had a high fixing power over time. The cosmetic feel of the hair was soft. Removal on shampooing was good.

What is claimed is:

1. A cosmetic composition packaged in an aerosol device comprising, in a cosmetically acceptable medium, at least one film-forming anionic polyurethane elastomer (A) and at least one propellant, wherein the at least one film-forming anionic polyurethane elastomer (A) comprises:
(i) at least one side chain comprising at least one ethylene oxide unit, wherein the at least one side chain
represents from 12 to 80% by weight of polyurethane (A)
has a Mw ranging from 1000 g/mol to 30,000 g/mol
(ii) one main chain comprising at least one unit derived from:
a polypropylene glycol (PPG) and optionally a second non-ionic polyol,
a dihydroxy-carboxylic acid and
at least one di-isocyanate, and optionally
a chain extender organic polyamine having an average of about at least two primary amine groups.

2. The composition according to claim 1, wherein the at least one film-forming anionic polyurethane elastomer (A) is chosen so that the film obtained by drying the polyurethane elastomer (A), at ambient temperature (24° C.±2° C.) and at a relative humidity of 48%±5%, exhibits a mechanical profile defined by at least:
(a) a degree of elongation at break (εᵣ) of greater than or equal to 730%±5%; and/or
(b) an instantaneous recovery (Rᵢ) of greater than or equal to 70%±5%, after an elongation of 150%; and/or
(c) a recovery (R₃₀₀ₛ) at 300 seconds of greater than 80%±5%, and the polyurethane elastomer (A) being soluble or dispersible, either in an aqueous medium or in a mixture of water/ethanol solvents comprising at most 30% by weight of ethanol.

3. The composition according to claim 2, wherein the concentration of the at least one film-forming anionic polyurethane elastomer (A) ranges from 0.05% to 20% by weight relative to the total weight of the composition.

4. The composition according to claim 3, wherein the concentration of the at least one film-forming anionic polyurethane elastomer (A) ranges from 0.1% to 15% by weight relative to the total weight of the composition.

5. The composition according to claim 3, wherein the concentration of the at least one film-forming anionic polyurethane elastomer (A) ranges from 1% to 10% by weight relative to the total weight of the composition.

6. The composition according to claim 2, wherein the at least one propellant is chosen from dimethyl ether, C₃₋₅ alkanes, chlorinated and fluorinated hydrocarbons, carbon dioxide gas, nitrous oxide, nitrogen, and compressed air.

7. The composition according to claim 6, wherein the at least one propellant is chosen from dimethyl ether, and C₃₋₅ alkanes.

8. The composition according to claim 2, wherein the cosmetically acceptable medium is a liquid phase and the liquid phase/propellant ratio by weight ranges from 50 to 0.05.

9. The composition according to claim 8, wherein the liquid phase/propellant ratio by weight ranges from 50 to 1.

10. The composition according to claim 2, wherein the cosmetically acceptable medium comprises at least one solvent chosen from water, C₁-C₄ alcohols, propylene carbonate, polyols, polyol ethers, and acetone.

11. The composition according to claim 10, wherein the cosmetically acceptable medium comprises ethanol.

12. The composition according to claim 10, wherein the cosmetically acceptable medium is aqueous or aqueous/alcoholic, wherein the alcohol is a C₁-C₄ alcohol.

13. The composition according to claim 2, further comprising at least one agent chosen from a plasticizing agent, a cosmetic agent and an agent which facilitates the formation of a film of the at least one film-forming anionic polyurethane elastomer (A) on keratinous substances.

14. The composition according to claim 2, further comprising at least one adjuvant chosen from silicones, in the soluble, dispersed and microdispersed form, other styling polymers, ceramides and pseudoceramides, vitamins and provitamins, water-soluble and fat-soluble and silicone-comprising and non-silicone-comprising sunscreens, pearlescent and opacifying agents, sequestering agents, antifoaming agents, conditioning agents, cations, cationic surfactants, fatty alcohols, silicone gums and resins, chitosans and their derivatives, solubilizing agents, acidifying agents, basifying agents, inorganic and organic thickening agents, antioxidants, hydroxy acids, penetrating agents, fragrances, peptizing agents, amino acids and preservatives.

15. The composition according to claim 14, wherein the provitamin is panthenol.

16. The composition according to claim 14, wherein the conditioning agents are cationic polymers.

17. The composition according to claim 14, wherein the inorganic and organic thickening agents are natural or synthetic nonionic associative thickening agents.

18. A cosmetic method for shaping the hair, comprising:
applying to the hair a cosmetic composition packaged in an aerosol device,
wherein the cosmetic composition comprises, in a cosmetically acceptable medium, at least one film-forming anionic polyurethane elastomer (A) and at least one propellant, wherein the at least one film-forming anionic polyurethane elastomer (A) comprises:
(i) at least one side chain comprising at least one ethylene oxide unit, wherein the at least one side chain
represents from 12 to 80% by weight of polyurethane (A)
has a Mw ranging from 1000 g/mol to 30,000 g/mol
(ii) one main chain comprising units derived from:
a polypropylene glycol (PPG) and optionally a second non-ionic polyol;
a dihydroxy-carboxylic acid and
at least one di-isocyanate, and optionally
a chain extender organic polyamine having an average of about at least two primary amine groups,
and wherein the at least one film-forming anionic polyurethane elastomer (A) is chosen so that the film obtained by drying the polyurethane elastomer (A), at ambient temperature (24° C.±2° C.) and at a relative humidity of 48%±5%, exhibits a mechanical profile defined by at least:
(a) a degree of elongation at break (εᵣ) of greater than or equal to 730%±5%; and/or
(b) an instantaneous recovery (Rᵢ) at least equal to 70%±5%, after an elongation of 150%; and/or
(c) a recovery (R₃₀₀ₛ) at 300 seconds of greater than 80%±5%, and
the polyurethane elastomer (A) being soluble or dispersible, either in an aqueous medium or in a mixture of water/ethanol solvents comprising at most 30% by weight of ethanol.
